(19) United States
(12) Patent Application Publication
(10) Pub. No.: US 2005/0282870 A1
(43) Pub. Date: Dec. 22, 2005

(54) Peripherally-acting vasodilators

(76) Inventors: Stephen G. Carter, Andover, MA (US); Zhen Zhu, Tewksbury, MA (US); Kanu Patel, Derry, NH (US)

Correspondence Address: NIELDS & LEMACK, 176 EAST MAIN STREET, SUITE 7, WESTBORO, MA, 01581, US

(21) Appl. No.: 10/958,530

(22) Filed: Oct. 5, 2004

Related U.S. Application Data
(60) Provisional application No. 60/580,051, filed on Jun. 16, 2004.

(51) Int. Cl. A61K 31/455; A61K 31/198

(52) U.S. Cl. 514/355; 514/563

(57) ABSTRACT

A system for the delivery of chemical vasodilators into the skin for the purpose of increasing the blood flow in a tissue that exhibits compromised circulation. The designated purpose of this increased blood flow is to transiently reverse or inhibit or prevent the onset of an associated medical affliction that may in part or in whole, be either the consequence of that medical condition or participating in the clinical symptoms and etiology of that medical condition. The process and the effect of the topical delivery of a chemical vasodilator has been previously demonstrated for other medical and pharmaceutical applications, however the specific use of this class of drug molecules for the focused effect of improving blood flow to the skin and surrounding tissue with the goal of improving the metabolic health of the tissue and to possibly affect the symptoms of a medical condition is the specific aim of this patent.

This application claims priority of U.S. Provisional Application No. 60/580,051 filed Jun. 16, 2004, the disclosure of which is incorporated herein by reference.

### BACKGROUND OF THE INVENTION

Prior art describes that chemical vasodilators may be topically applied with the result being a physiological response of dilation of the skin and a resultant increase in local tissue blood flow. Those systems function by using a variety of penetration enhancer chemicals to assist the passive penetration of drug molecules through the stratum corneum layer of the skin into the dermal layer of the skin. Once in the dermal layer of skin, vasodilators act to dilate the capillaries and other blood vessels resulting in an increase in blood flow to the area.

This invention describes the composition of a topical drug formulation designed to rapidly absorb different types of a single or multiple set of vasodilators into the skin to the target tissues of different blood vessels, with or without the aid of external apparatus and with or without the use of a physical or molecular patch device or system, which may be used in some instances as a facilitator of the delivery of the vasodilators and/or also used as a functional reservoir for the vasodilator.

### SUMMARY OF THE INVENTION

The initial focused use of topically-applied and delivered peripheral vasodilators described in this invention is by individuals suffering from various types of peripheral vascular disease, and neuropathies, including those afflicted with type 1 and/or type 2 diabetes mellitus, neurotropic viral infections and stroke. Individuals suffering from these conditions can eventually experience the medical complications associated with the long-term deprivation of optimal blood flow, and the consequent decrease of nutrients and oxygen and removal of waste products. This diminished vascular capacity may result in an increased incidence of different forms of neuropathy, and then subsequently various forms of complications from the neuropathy, including a loss of sensation to the feet, resulting in abnormal pressures applied to the feet when walking, which in turn may lead to foot ulcers and in some cases eventually leading to partial or complete amputations.

The application of topical vasodilators in a form that would permit and promote dermal penetration and consequent vasodilation in the dermal tissue through multiple daily administrations, would offer the clinical and the patient communities the opportunity to effectively and directly address this problem.

The efficiency and the focused application of use by the present invention includes a broader range of drugs and agents that can be delivered topically and also addresses the use of this treatment for the medical conditions of neuropathy.

### DETAILED DESCRIPTION OF THE INVENTION

The invention describes the treatment of neuropathy and neuropathic disease conditions and complications arising from compromised circulatory problems with a topical vehicle containing one or more chemical vasodilators. The vehicle contains a mixture of chemical substances designed to assist in the penetration of the other components of the vehicle, including the vasodilators, into the dermal layer of the skin for the purpose of dilating the local capillary blood vessels. Dilation of the local blood vessels will result in an increase in blood flow to the treated area of skin. The transiently improved blood flow to the treated area should improve the metabolic condition of the tissue, which in turn should result in a healthier condition for nerve and associated tissue function.

There are published reports suggesting that in many medical conditions related to neuropathy, including diabetic neuropathy, there is a compromised microcirculatory environment around the nerves, which may have an influence on the normal functions of the nerves, which may lead to the abnormal sensations or lack of sensations noted with these medical conditions.

Improving the metabolic state around the nerve cells in the skin through an improved blood flow, which would increase the oxygenation and nutrient delivery to the tissue as well as improve the removal of cellular and tissue waste products, should improve the health and functioning of the nerve cells.

There are many different types of vasodilators that are candidates as a single mode therapeutic or in combination with other vasodilators, which may act biochemically different to induce vasodilation. The invention includes all chemicals that may be a direct vasodilator or those substances that serve as precursors or second messenger chemicals to induce or enhance the production of vasodilatory substance from the tissue and cell.

Examples of chemical vasodilators to be used in the delivery system include, by example only but are not limited to: amrinone, L-arginine, bamethan sulphate, bencyclane fumarate, benfurodil hemisuccinate, benzyl nicotinate, buflomedil hydrochloride, buphenine hydrochloride, butalamine hydrochloride, cetiedil citrate, ciclonicate, cinepazide maleate, cyclandelate, di-isopropylammonium dichloroacetate, ethyl nicotinate, hepronicate, hexyl nicotinate, ifenprodil tartrate, inositol nicotinate, isoxsuprine hydrochloride, kallidinogenase, methyl nicotinate, naftidrofuryl oxalate, nicametate citrate, niceritrol, nicoboxil, nicofuranose, nicotinyl alcohol, nicotinyl alcohol tartrate, nitric oxide, nonivamide, oxpentifylline, papaverine, papaveroline, pentifylline, peroxynitrite, pinacidil, pipratecol, propentofyltine, raubasine, suloctidil, teasuprine, thymoxamine hydrochloride, tocopherol nicotinate, tolazoline, xanthinol nicotinate, diazoxide, hydralazine, minoxidil, and sodium nitroprusside. Centrally acting agents include clonidine, quanaberz, and methyl dopa. Alpha-adrenoceptor blocking agents include indoramin, phenoxybenzamine, phentolamine, and prazosin. Adrenergic neuron blocking agents include bedmidine, debrisoquine, and guanethidine. ACE inhibitors include benazepril, captopril, cilazapril, enalapril, fosinopril, lisinopril, perindopril, quinapril, and ramipril. Ganglion-blocking agents include pentolinium and trimetaphan. Calcium channel blockers include amlodipine, diltiazem, felodipine, isradipine, nicardipine, nifedipine, nimodipine, and verapamil. Prostaglandins including: prostacyclin, thrombuxane A2, leukotrienes, PGA, PGA1, PGA2, PGE1, PGE2, PGD, PGG, and PGH. Angiotensin II analogs include saralasin. Other vasodilators include nitroglycerin, labetalol, thrazide, isosorbide dinitrate, pentaerythritol tetranitrate, digitalis, hydralazine, diazoxide, and sodium nitroprusside. Typically the vasodilator, used either as a single agent or in concert with other vasodilators is present in the topical vehicle at concentration between 0.0005% to 15%, depending on the specific vasodilator used and the pharmacologic properties of the chemical. This element may serve exclusively as the vasodilation agent or it may also, in addition, serve another function to the delivery complex such as penetration, as the active drug agent, or binding of the delivery complex. One or more vasodilators or chemically modified vasodilators can be used in the delivery complex at any one time for one formulation for the purpose of transdermally delivering an active drug molecule or agent

The delivery complex will also include chemicals that function as a penetration enhancers to assist in the transport of the vasodilators from the skin surface, through the stratum corneum and into the dermal layer of the skin. Suitable enhancers include by example only but are not limited to: individual fatty acids, fatty acid esters, polyols, amides, various anionic, cationic and nonionic surfactants such as but not limited to sodium laurate and sodium lauryl sulfate, phospholipids, cholesterol and cholesterol derivatives, m-pyrrole, dimethyl acetamide, limonene, sphingolipids, ceramides, terpenes, alkanones, menthol, various organic acids, such as but not limited to salicylic acid, citric and succininc acid, prostaglandins, decyl methyl sulfoxide, urea, sulfoxide alcohols, plant extract oils. Suitable fatty acids include by example but are not limited to: linoleic acids, linolenic acids, oleic acids, stearic acids, and myristic acids. Phospholipids include by example but are not limited to: phosphatidylcholine, phosphatidylethanolamine, and phosphatidylserine. Plant extract oils include peanut oil, hemp, barag, olive oil, sunflower oil, soybean oil, monoi oil and macadamia oil, with olive oil being preferred. Suitable alcohols for the plant extract oil/alcohol mix include ethyl alcohol, isopropyl alcohol, methyl alcohol and witch hazel. Olive oil mixed with isopropyl alcohol is a preferred vegetable oil/alcohol mix. Eucalyptol is a further suitable example of a vegetable oil/alcohol mix. Suitable ratios of vegetable oil: alcohol range from about 5:1 to about 1:10, preferably 1:2. Suitable amounts of plant extract oil or plant extract oil/alcohol mix in the delivery complex range from about 1% to about 66% by weight, more preferably from about 10% to about 33.3% by weight. This component may serve exclusively as the penetrating agent or it may also, in addition, serve another function to the delivery complex such as vasodilation, as the active drug agent, or binding of the delivery complex. One or more penetrating agents or chemically modified penetrating agents may be used in varying quantities or ratios with respect to the other component parts in the drug delivery complex at any one time. The penetrating agent molecule may also serve in any of the other critical functions for the delivery system, including that of active drug molecule, vasodilator, and/or polymer.

The delivery complex may or may not include a polymer or a formulation stabilizer molecule. This substance is designed to be compatible with the composition of the remainder of the chemicals in the formulation and typically to be an inert functional component that will be degraded or non-penetrating into the skin. Examples of suitable polymers or binding agents include but are not limited to: carbopol, hydroxyethylcellulose, u-care polymer, and water-soluble gums (e.g., agar, arabic, carob, CMC, carrageenans, ghatti, guar, karaya, kadaya, locust bean, tragacanth, and xanthan gums). The polymer should be used in an amount ranging from about 1% to about 20% by weight, most preferably an amount equal to the amount of active ingredient used. Exceptions may be made to this statement if the polymer is serving both the function of the polymer as well as the active drug molecule or agent or in the function of vasodilator and/or in the function of the penetration enhancer. The polymer may serve exclusively as the polymer or it may also, in addition, serve another function to the delivery complex such as vasodilation, penetration, or as the active drug agent of the delivery complex.

The preparation of the vasodilator-containing topical vehicle may be achieved through standard formulation processes, depending on the chemical characteristics of the specific components. Typically, a hydrogel or aqueous or non-aqueous emulsion is prepared by first preparing a mixture of the water-soluble chemicals and mixing until homogenous, then blending, through a slow addition of the non-aqueous phase of the mixture until homogeneity is achieved.

Although the delivery complex is typically capable of transporting the drug molecules through the skin efficiently without the use of a patch device, in some instances a patch or patch-like device may be desirable. Patches, pre-impregnated with a component of the delivery complex or with the complete delivery complex may be preferable for the delivery of doses of vasodilators. If a patch is used in conjunction with the delivery complex of the present invention, the patch may be a non-breathable layer on which the active ingredient is placed. Suitable non-breathable layers include sheets of plastic, polyethylene, polyvinyl chloride, wax paper, foil, latex, etc., and combinations thereof. Those skilled in the art will recognize that any non-breathable substance that is not deleterious to the active drug ingredient or to the other delivery system component parts being used, and that does not cause any irritation upon contact with skin, may be used. The non-breathable layer functions to create and control a suitable microenvironment at the administration site to facilitate the delivery of drugs across the skin and into the bodily fluids. Too cold an environment can result in little blood supply to the dermal barrier; pores and other natural openings in the dermal barrier constrict, thereby preventing efficient transport. Too hot an environment can enhance secretion and perspiration and vapor flow through the dermal barrier, creating negative conditions for transdermal drug delivery activity. Too dry an environment can cause an element or elements of the delivery complex to evaporate quickly, losing its ability to transport. The enhanced evaporation also creates negative transport pressure. Too humid an environment can cause dilution of the active ingredient, diminishing the capacity of the active ingredient and also creating negative transport activity. The non-breathable layer or patch may potentially maintain more desirable conditions (e.g., temperature, humidity, pore size, enhanced localized blood flow) for transdermal drug transportation at the drug delivery site. If desired, the patch or non-breathable layer can be secured to the skin by any suitable means, such as with a bandage with adhesive or fasteners. In the preferred configuration, no adhesive is used; instead compression is used as discussed in detail below.

The critical function of vasodilation in the invention may be further enhanced when other conditions are applied to the area of skin being used to administer the active drug. One of these enhanced conditions (but not limited to this example) is to increase the blood supply at the transport site, using compression. Specifically, the non-breathable layer can be applied to the skin tightly, such as with a tightly wrapped bandage. Preferably the compression at the transport site is greater than zero pounds per square inch but less than about 10 pounds per square inch. Too much compression can result in restricted blood supply. The positive pressure applied also aids in moving the delivery complex through the dermal layer and into eventual contact with the blood supply.

The magnitude of a prophylactic or therapeutic dose of the formulation of the present invention may vary with the severity and nature of the condition to be treated and the route of administration. The dose and the frequency of the dosing may also vary according to the age, body weight and response of the individual patient. In general, 1-5 grams of the formulation per foot is desired, and is preferably applied 1-3 times each day for at least one month. The terms “a therapeutically effective amount” and “an amount sufficient to treat the disorder but insufficient to cause adverse effects” are encompassed by the above-described dosage amounts and dose frequency schedule.

### FORMULATION EXAMPLE 1

| | Weight by % |
|---|---|
| Cellulosize QP 5200 H | 1.000 |
| Methyl Nicotinate | 1.000 |
| Distilled water | 98.000 |

### FORMULATION EXAMPLE 2

| | Weight by % |
|---|---|
| 0.1% Tolazoline in DMA | 10 |
| Ucare JR 30 M | 1 |
| Propylene Glycol | 89 |

### FORMULATION EXAMPLE 3

| | Weight by % |
|---|---|
| Petrolatum | 7.000 |
| Vitamin E nicotinate | 0.100 |
| Propyl Paraben | 0.100 |
| Cetyl Alcohol | 5.000 |
| Lipomulse 165 | 3.000 |
| Glycerin | 2.000 |
| Propylene Glycol | 1.000 |
| Methyl paraben | 0.200 |
| DMDM Hydantoin | 0.250 |
| L-Arginine | 2.000 |
| Methyl nicotinate 1% solution | 2.500 |
| Pemulin TR-1 | 0.300 |
| Water | 76.550 |

### EXAMPLE 4

Vasodilation induced by topical application of methyl nicotinate was evaluated and compared with the vasodilatory response to acetylcholine and sodium nitroprusside in healthy subjects and diabetic neuropathic patients. Ten diabetic patients with peripheral neuropathy and 10 age- and sex-matched healthy control subjects were enrolled. The vasodilatory response to topical application of 1% methyl nicotinate and a placebo emulsion at the forearm and dorsum of the foot skin at 4, 15, 30, 60 and 120 min was measured using Laser Doppler Perfusion Imaging. The vasodilatory response to iontophoresis of 1% acetylcholine and 1% sodium nitroprusside solutions was also evaluated.

The maximal vasodilatory response to acetylcholine, sodium nitroprusside and methyl nicotinate was similar at the forearm and foot level in the diabetic patients. In the control group, the responses to acetylcholine, sodium nitroprusside and methyl nicotinate were similar on the forearm but in the foot, the methyl nicotinate vasodilatory response was higher when compared to the acetylcholine and sodium nitroprusside responses. Methyl nicotinate-related vasodilation was present 5 min after the application, reached its peak at 15-30 min and declined to pre-application levels 120 min afterward. See “Topical methyl nicotinate-induced skin vasodilation in diabetic neuropathy”, Journal of Diabetes and Its Complications, 17 (2003) 205-210, the disclosure of which is hereby incorporated by reference.

1. A method of treating or preventing neuropathy in a patient, comprising topically administering to said patient an effective amount of a formulation comprising at least one vasodilator along with a penetration enhancer.

2. The method of claim 1, wherein said formulation is administered locally to the foot of said patient.

3. The method of claim 1, wherein said formulation is administered locally to the forearm of said patient.
